(19) United States
(12) Patent Application Publication
(10) Pub. No.: US 2023/0047813 A1
(43) Pub. Date: Feb. 16, 2023

(54) COSMETIC COMPOSITIONS AND METHODS

(71) Applicant: ELC Management LLC, Melville, NY (US)

(72) Inventors: John F Logalbo, Dix Hills, NY (US); LeighAnn Mulholland, Sayville, NY (US); M Megan Tzakas, Garden City South, NY (US); Doug Melenkevitz, Boonton, NJ (US); Patrick Block, Deer Park, NY (US); Tao Zheng, Dix Hills, NY (US)

(21) Appl. No.: 17/873,434

(22) Filed: Jul. 26, 2022

Related U.S. Application Data
(60) Provisional application No. 63/226,710, filed on Jul. 28, 2021.

(51) Int. Cl. A61K 8/02; A61K 8/67; A61K 8/90; A61K 8/96; A61K 8/25; A61K 8/81; A61Q 1/06

(52) U.S. Cl. CPC A61K 8/0229; A61K 8/678; A61K 8/90; A61K 8/965; A61K 8/25; A61K 8/8129; A61Q 1/06; A61K 2800/43; A61K 2800/522; A61K 2800/95

(57) ABSTRACT

A method for processing a high pigment loaded cosmetic composition may provide a pre-dispersion phase, a grind phase, and a full extender phase. The method of processing the cosmetic composition may fully saturate and disperse solid particulates of at least 35% by weight of the cosmetic composition. The method for processing the cosmetic composition may provide a high color payoff in a single swipe or application. Further, the method for processing the cosmetic composition may reduce feathering and bleeding even with a high pigment load.

### CROSS-REFERENCE TO RELATED APPLICATION

The present Application is a non-provisional of, and claims priority to, U.S. Patent Application Ser. No. 63/226,710, filed Jul. 28, 2021, the disclosure of which is incorporated by reference in its entirety.

### FIELD OF THE DISCLOSURE

The present disclosure generally relates to processing a cosmetic composition having a high pigment load, and more particularly, processing a high-pigment loaded cosmetic composition having long-wear attributes.

### BACKGROUND

Cosmetic compositions that provide high pigment loads can be difficult to process. Conventional cosmetic compositions typically sacrifice moisturization and long wear attributes when high pigment loads are incorporated into the composition. Additionally, conventional cosmetic compositions can fail to sufficiently saturate pigments, which may be powder, and can fail to fully disperse pigments within the entirety of the cosmetic composition. Based on the foregoing, there is an ongoing need for a cosmetic composition and processing method thereof which can deliver a high pigment load without the drawbacks of limited wear, dryness, and low payoff.

### SUMMARY

Embodiments of the present disclosure generally provide a high pigment loaded cosmetic composition and processing method thereof. Embodiments of the present disclosure may provide a cosmetic composition with comfort and long-wear attributes. The composition according to embodiments of the present disclosure may provide high-color payoff in one swipe given high pigment loads for color-saturated shades. It may be long-wearing, non-feathering, non-bleeding, non-fading, and may provide immediate and long-term moisturization. Optimized pigment saturation in combination with a natural film former plus a wax complex blend may provide comfort on application and high-performance comfortable wear.

The cosmetic composition may be in a plurality of product forms including, but not limited to, blush, eyeshadow, face powder, lipstick, lip gloss, concealers, hot pour emulsions, anhydrous hot pour systems, and other similar compositions. These product forms can be utilized in sun protection factor (SPF) product forms in embodiments of the present disclosure. These product forms may deliver high levels of pigment and powders that may provide a one swipe ultra-saturated color deposit. These compositions may have a lightweight feel, and may be non-caking, non-pilling, and nondrying with comfort. The method of processing may be applicable to product forms including, but not limited to, a blush, eyeshadow, face powder, lipstick, highlighting, SPF forms, and other similar compositions. Further, the cosmetic composition and method thereof may be provided in product forms including, but not limited to, pencils, sticks, pots, and other similar compositions.

Embodiments of the present disclosure may provide a method of making a cosmetic composition comprising mixing, in a pre-dispersion phase, at least 35% by weight of the cosmetic composition of one or more solid particulates with liquid ingredients, wherein the one or more solid particulates comprise pigments, powders, and fillers, and wherein the liquid ingredients include approximately 29-42% by weight of the cosmetic composition of one or more emollients and 0.25-5% by weight of the cosmetic composition of one or more wetting agents. The liquid ingredients may further comprise 0.5-10% by weight of one or more film formers; 0.5-1% by weight of vitamin E; 2-3% by weight of a copolymer block; 1-2% by weight of clay; and 1-2% by weight of silica. The one or more solid particulates may fully saturate the liquid ingredients, and the one or more solid particulates may fully disperse in forming the cosmetic composition. The method also may include grinding, in a grind phase, the cosmetic composition until particles have less than approximately 12 microns or approximately 7 Hegman units. The method may further comprise mixing, in a full extender phase, the ground cosmetic composition with one or more of the following: one or more emollients, one or more wetting agents, one or more waxes, one or more film formers, and one or more moisturizers. The method also may comprise heating the cosmetic composition up to approximately 95 degrees Celsius.

Other embodiments of the present disclosure may provide a cosmetic composition, comprising: one or more solid particulates; at least one of a dispersing agent, a wetting agent, or a mixture thereof; at least one film former; and at least one main carrier. The composition may comprise approximately 35-50% by weight of the one or more solid particulates; approximately 0.25-5% by weight of the at least one of the dispersing agent, the wetting agent, or the mixture thereof; approximately 0.5-10% by weight of the at least one film former; and approximately 35% by weight of the at least one main carrier. The composition may further comprise at least 35% by weight of the one or more solid particulates; at least 0.25% by weight of the at least one of the dispersing agent, the wetting agent, or the mixture thereof; at least 0.50% by weight of the at least one film former; at least 5% by weight of one or more wax-related components; and at least 0.25% by weight of one or more moisturizing agents. The one or more solid particulates may be selected from the group consisting of pigments, powders, fillers, and mixtures thereof. The one or more solid particulates may be surface treated to provide a shear-resistant composition. The main carrier may be selected from one or more of long-chain fatty alcohol liquids, oils, esters, and emollients. The cosmetic composition may provide ultra-saturated color deposition when applied to a user, wherein the ultra-saturated color deposition may provide at least 70% opacity.

### DETAILED DESCRIPTION

Embodiments of the present disclosure generally provide a high-pigment loaded cosmetic composition having comfort and long-wear attributes and processing method thereof. Compositions according to embodiments of the present disclosure may provide high-color payoff in one swipe. The compositions may be long-wearing, non-feathering, non-bleeding, non-fading, and may provide immediate and long-term moisturization. Optimized pigment saturation in combination with a natural film former plus a wax complex blend may provide comfort on application and high-performance comfortable wear.

Compositions according to embodiments of the present disclosure may include solid particulates, which may be pigments, powders, fillers, and/or mixtures thereof, at levels between 20-50%, more preferably 30-50%, and at least 35%.

The cosmetic composition according to embodiments of the present disclosure may provide a high pigment load. It should be appreciated that a high pigment load may be at least 45% by weight of the composition in some embodiments of the present disclosure. It should also be appreciated that a high pigment load may be less than or greater than 45% by weight of the cosmetic composition without departing from the present disclosure.

The particulate materials may comprise various organic and/or inorganic pigments. The organic pigments may generally be various aromatic types including, but not limited to, azo, indigoid, triphenylmethane, anthroquinone, and xanthine dyes which are designated as D&C and FD&C blues, browns, greens, oranges, reds, yellows, etc. Organic pigments generally may include insoluble metallic salts of certified color additives, referred to as the Lakes. Inorganic pigments may include iron oxides, ultramarines, chromium, chromium hydroxide colors, and mixtures thereof. Iron oxides of red, blue, yellow, brown, black, and mixtures thereof may be used.

In a case where the composition may comprise mixtures of pigments and powders, suitable ranges include about 0.01-75% pigment and 0.1-75% powder, such weights by weight of the total composition. The pigment can be surface treated with compounds including, but not limited to, isopropyl titanium triisostearate (ITT), trityl tetrazole bromomethyl biphenyl (TTBB2), silane, and/or dimethicone. Powders and fillers can be selected from compounds including, but not limited to, bentonite hectorite, silica, or BPD-500, mica, and/or barium sulfate.

Particulate materials may be colored or non-colored (for example, white) non-pigmented powders. Suitable non-pigmented powders include, but are not limited to, bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, and/or mixtures thereof. The above-mentioned powders may be surface treated with compounds including, but not limited to, lecithin, amino acids, mineral oil, silicone, and/or various other agents either alone or in combination, which may coat the powder surface and render the particles more lipophilic in nature.

Compositions according to embodiments of the present disclosure may include one or more dispersants that can be added independently of the solid particles or in the form of colloidal dispersion of particles. A dispersing agent may protect the dispersed particles against their agglomeration or flocculation. The concentration of dispersant generally used to disperse satisfactorily solid particles (without flocculation) and, in particular, to stabilize a colloidal dispersion of the particles, may range from 0.3 to 5 mg/m to 2, preferably from 0.5 to 4 mg/m to 2, of the surface of particles. This dispersant may be a surfactant, an oligomer, a polymer, or a mixture thereof, carrying one or more functionalities having a high affinity for the surface of the particles to disperse. In particular, they may be physically or chemically attached to the surface of the particles to disperse. These dispersants may have, in addition, at least one functional group compatible or soluble in the continuous medium. In particular, use may be made of the esters of the polyhydroxy acid-12 stearic acid such as the stearate of poly (12-hydroxystearic) of molecular weight of about 750 g/mol, such as that sold under the name Solsperse 21,000 by Avecia, esters of polyhydroxy-12-stearic acid with polyols such as glycerol, diglycerin, such as the polyglyceryl-2 dipolyhydroxystearate (CTFA name) sold under the reference Dehymuls PGPH by the company HENKEL (or poly (12-hydroxystearate)diglycerol), or alternatively poly (12-hydroxystearic) such as that sold under the reference Arlacel P100 by Unigema, and mixtures thereof.

Other dispersants that may be used in the composition according to embodiments of the present disclosure may include, but are not limited to, quaternary ammonium derivatives of polycondensed fatty acids 17,000 sold by Avecia, and/or mixtures of polydimethylsiloxane/oxypropylene such as those sold by the company Dow Corning under the references DC2-5185, DC2-5225 the C. Poly12-dihydroxystearic acid and esters of the polyhydroxy12-stearic acid may be used in a hydrocarbon or fluorinated medium, whereas the oxyethylene/oxypropylenated dimethylsiloxane mixtures may be used for a silicone medium.

At least one other dispersing and wetting agent may be included at levels between 0.25-5% in embodiments of the present disclosure. This may include surfactants including, but not limited to, polyglyceryl- 3 diisostearate, polyglyceryl- 6 polyricinoleate, lecithin, sorbitan sesqueoleate, and/or polyhydroxystearic acid.

Film formers may be included at levels between 0.50-10% and may be selected from compounds including, but not limited to, dextrin isostearate/isostearic acid, and/or PPG-51/SMDI copolymer. Dextrin isostearate may exhibit wetting and color intensifying properties in addition to its film-forming capabilities.

Film forming polymers may include, for example, organosiloxane resins comprising combinations of R3S1O1/2 “M” units, R2S1O “D” units, RS1O3/2 “T” units, S102 “Q” units in ratios to each other that satisfy the relationship RnSiO(4_ny2) where n is a value between 1.0 and 1.50 and R is methyl. In some embodiments of the present disclosure, a small amount, up to 5%, of silanol or alkoxy functionality may also be present in the resin structure as a result of processing. The organosiloxane resins must be solid at about 25° C. and have a molecular weight range of from about 1,000 to about 10,000 grams/mole. The resin may be soluble in organic solvents such as toluene, xylene, isoparaffins, and cyclosiloxanes or the volatile carrier, indicating that the resin is not sufficiently crosslinked such that the resin is insoluble in the volatile carrier. Particularly preferred are resins comprising repeating monofunctional or R3SiOiy2 “M” units and the quadrofunctional or S102 “Q” units, otherwise known as “MQ” resins as disclosed in U.S. Pat. No. 5,330,747, Krzysik, issued Jul. 19, 1994, incorporated herein by reference. In the present disclosure, the ratio of the “M” to “Q” functional units may be preferably about 0.7, and the value of n may be 1.2. Organosiloxane resins as described above are commercially available such as trimethylsiloxysilicate/cyclomethicone D5 Blend available from GE Toshiba Silicone, Wacker 803 and 804 available from Wacker Silicones Corporation of Adrian Mich., KP545 from Shin-Etsu Chemical and G. E. 1170-002 from the General Electric Company. In the present disclosure, by having a film-forming polymer mainly in the second layer, the film-forming polymer may exist in a higher concentration at a localized area, thereby forming a film of higher film intensity when applied to the skin, compared to the remainder of the composition. Such concentrated area of high film intensity may provide improved adhesion of the entire composition to the skin. Namely, by providing the film-forming polymer mainly in the second layer, the amount of film-forming polymer included in the entire composition can be reduced, or if the same amount of film-forming polymer is formulated in the second layer, an entire composition having improved adhesion may be obtained. In a preferred embodiment, the content level of film-forming polymer in the second layer may be from about 0.1% to about 20%, preferably from about 0.5% to about 10%, and more preferably from about 1% to about 8%.

Main carriers may include, but are not limited to, long-chain fatty alcohol liquids such as octyldodecanol, oleyl alcohol, and/or squalene. Oils such as but not limited to castor oil, caprylic/capric triglyceride, jojoba oil, and/or hydrogenated variants may be included. Esters which include and not limited to alkyl esters, isononyl isononoate, cetearyl isononanoate and/or ethylhexyl isononanoate may be included in compositions according to embodiments of the present disclosure. Emollients such as, but not limited to, olive squalane, and/or natural butters including shea butter and/or kokum butter also may be incorporated into compositions according to embodiments of the present disclosure.

In a stick form, compositions according to embodiments of the present disclosure may include structural agents at levels between 5.0-20% such as, but not limited to, paraffinic waxes including microcrystalline, polyethylene, beeswax, and gel stabilizers such as, but not limited to, butyl stearate/butylene/ethylene, styrene, copoly/ethylene/propylene/styrene, and/or copolymer/dibutyl lauroyl glutamide. Optional components may include anti-oxidants such as but not limited to tocopheryl acetate and/or butylated hydroxytoluene (BHT); and/or moisturizers between ranges of 0.25-5% such as, but not limited to, sodium hyaluronate/hydrogenated castor oil and/or phytosteryl/octyldodecyl lauroyl glutamate. Processing this formulation type may include incorporation of a high percentage of the formulas liquid base into the grind. This, in combination with a higher percentage of multiple wetting agents (surfactants), may enable full pigment/powder saturation and development in embodiments of the present disclosure.

A preferred procedure for preparing cosmetic compositions according to embodiments of the present disclosure may include a pre-dispersion phase, a grind phase, and a formula extender phase, as described herein.

Pre-Dispersion Phase

TABLE 1
Cosmetic Composition Exemplary Formula

| | Formula A | Formula B | Formula C |
|---|---|---|---|
| Ingredients | Wt % | Wt % | Wt % |
| Pigment | 46.76 | 54.10 | 56.71 |
| Squalane¹ | 5.84 | 2.02 | 2.12 |
| Octyldodecanol² | 35.45 | 30.41 | 27.04 |
| PPG-51/SMDI Copolymer³ | 3.50 | 4.05 | 4.24 |
| Polyglyceryl-3 Diisostearate⁴ | 2.33 | 2.36 | 2.47 |
| Polyglyceryl-6 | 0.87 | 1.00 | 1.05 |
| Polyricinoleate⁵ | | | |
| Vitamin E | 0.58 | 0.67 | 0.70 |
| Copolymer Block⁶ | 2.33 | 2.69 | 2.83 |
| Disteardimonium Hectorite⁷ | 1.17 | 1.35 | 1.42 |
| Silica⁸ | 1.17 | 1.35 | 1.42 |

¹Neossance ® Squalane, available from Centerchem Inc. Used as skin softener.
²Eutanol ® G, available from BASF Care Creations. Long fatty chain alcohol used as an emollient.
³Polyolprepolymer-14, available from Barnet Products Inc. and Penederm Inc. Copolymer of PPG-51 and saturated methylenediphenyldiisocyanate used for wear/conditioning.
⁴Plurol ® Diisotearique CG, available from Gattefossé. Used as surfactant.
⁵Hexaglyn PR-15, available from Nikko Chemicals Co., Ltd. Used as surfactant.
⁶Versagel ® S1000, available from Penreco ®. Versagel includes Hydrogenated Polyisobutene (And) Butyl Stearate (And) Isostearyl Alcohol (And) Butylene/Ethylene/Styrene Copolymer (And) Ethylene/Propylene/Styrene Copolymer (And) Dibutyl Lauroyl Glutamide.
⁷Bentone ® 38 VCG, available from Elementis. Used for adhesion.
⁸Spheron 20MB, available from Presperse Inc. Used for absorption.

Using a high shear disperser or mixer, such as a Cowles or Scott mixer, a first mixture including squalane, octyldodecanol, PPG-51/SMDI copolymer, polyglyceryl-3 diisostearate, polyglyceryl-6 polyricinoleate, and vitamin E may be mixed in a first auxiliary. The ingredients may be mixed at room temperature and yield uniform distribution.

Using a propeller mixer, octyldodecanol may be inserted in a second auxiliary. The mixer may be heated to approximately 950 Celsius, or up to 100 Celsius over the drop point of octyldodecanol used in embodiments of the present disclosure. The mixer may be turned on to an operating state. At approximately 95° Celsius, a copolymer block may be added to the second auxiliary to create a second mixture or premix. Mixing may occur for approximately 30 minutes or until the mixture is uniformly distributed and clear in color. It should be appreciated that the temperature may be up to 990 Celsius without departing from the present disclosure.

In the high shear disperser or mixer, the second mixture from the second auxiliary may be added to the first mixture in the first auxiliary. The first mixture and the second mixture may be mixed for approximately five minutes or until the composition is uniformly distributed.

Stepwise, disteardimonium hectorite and silica may then be added, mixing well to achieve a uniform distribution over a sufficient duration of time. A sweep and disperse pigment may be done within the composition for approximately 30 minutes. The mixer may then be stopped so that the composition may be scraped from the blades. The composition may then be mixed for approximately 10 additional minutes.

The ingredients of Table 1 may be mixed to adequately saturate and uniformly distribute pigment throughout the cosmetic composition. Air trapped between particles of the cosmetic composition may be filled with oil, thereby changing the refractive index.

Grind Phase

The composition may be transferred from the pre-dispersion phase to a ball mill via a pump. Using the ball mill (such as, by Fryma Karuma MS12), a grind phase may begin by effectively mixing the composition to produce a dispersion having particle size less than approximately 12 microns or a Hegman reading of approximately 7 or greater. It should be appreciated that the ball mill may have a rotor speed of approximately 1800 rates per minutes (RPM). It should also be appreciated that the flow rate of cosmetic composition in the ball mill may be approximately 300 to 1,000 grams per min (g/min). Bead material used in the ball mill may be zirconium oxide, which may be known as fused zirconium oxide or zirconia silica. The size of the bead material utilized in the ball mill may be approximately 0.8 to 1.0 millimeters (mm). It should be appreciated that a sufficient time to achieve a uniform mass of the premixed ingredients may be from approximately 15 minutes to 30 minutes in embodiments of the present disclosure. It should further be appreciated that the carrier and pigments selected may determine the time needed to sufficiently mix ingredients of the cosmetic composition. Pigment dispersions according to embodiments of the present disclosure may range from flowable to paste-like. There may be up to 60% pigment (20-60%) in the grind phase in some embodiments of the present disclosure.

Formula Extender Phase

TABLE 2
Cosmetic Composition Exemplary Formula

| | Formula A | Formula B | Formula C |
|---|---|---|---|
| Ingredients | Wt % | Wt % | Wt % |
| Composition Formed in | 85.00 | 73.50 | 70.10 |
| Grind Phase | | | |
| Squalane¹ | 0.0001 | 3.48 | 3.48 |
| Octyldodecanol² | 0.0001 | 7.77 | 11.17 |
| Polyglyceryl-3 Diisostearate⁴ | 0.0001 | 0.25 | 0.25 |
| Polyethylene⁹ | 6.00 | 6.00 | 6.00 |
| Microcrystalline Waxes¹⁰ | 5.00 | 5.00 | 5.00 |
| Dextrin Isostearate¹¹ | 3.00 | 3.00 | 3.00 |
| Phytosteryl/Octyldodecyl | 0.50 | 0.50 | 0.50 |
| Lauroyl Glutamate¹² | | | |
| Sodium Hyaluronat¹³ | 0.50 | 0.50 | 0.50 |

⁹Performalene ® PL, available from New Phase Technologies
¹⁰Multiwax W-835, available from Sonneborn LLC; Ozokerite Wax Pastilles 1021P, available from Strahl and Pitsch
¹¹Unifilma Hvy, available from Chiba Flour Milling
¹²Eldew PS-203, available from Ajinomoto Co., Inc.
¹³Hyacolor, available from Bloomage Biotechnology Corporation

The cosmetic composition formed in the grind phase may be transferred into a heated vessel or a conventional propeller mixer. It should be appreciated that a propeller mixer may be utilized for thin grinds while a sweep mixer may be utilized for thick grinds in embodiments of the present disclosure.

Squalane, octyldodecanol, and polyglyceryl-3 diisostearate may be added to the vessel and heated to approximately 950 Celsius. It should be appreciated that the temperature may be up to 99° Celsius without departing from the present disclosure.

At a temperature of approximately 800 Celsius, polyethylene and microcrystalline waxes may be added. It should be appreciated that the temperature may be greater than or less than 80° Celsius without departing from the present disclosure. Dextrin isostearate or a film former may then be added and mixed for approximately 15 minutes until the composition is uniformly distributed. Phytosteryl/octyldodecyl lauroyl glutamate and sodium hyaluronat may then be added. The composition may be mixed for approximately five minutes until the composition is uniformly distributed. The composition may be discharged and molded in a filler.

The cosmetic composition may be flowable and capable of being poured into a pot or stick form in embodiments of the present disclosure. It should be appreciated that the cosmetic composition may form emulsion-based systems in some embodiments of the present disclosure. It should also be appreciated the cosmetic composition may have any form including, but not limited to, an eye, cheek, face, lip, and/or other product forms.

The cosmetic composition may provide an ultra-saturated color deposition. For example, a single application, swipe, or use of the cosmetic composition may provide a pigment load high enough to remain on a wearer for up to approximately 12 hours. The ultra-saturated color deposition may provide a long wearing cosmetic composition that may not be inadvertently removed through normal activities. Normal activities may include, but are not limited to, eating, drinking, and/or rubbing against a fabric. The cosmetic composition may provide pigment with a viscosity up to 96,000 centipoise (cps). It should be appreciated that the viscosity of pigment may be greater than or less than 96,000 cps without departing from the present disclosure.

Opacity Test Measurement

Opacity may be evaluated by a drawdown method using a 2 mil drawdown bar over a black and white Leneta chart. An average of five drawdowns may be performed for each shade or pigment color. The opacity of the cosmetic composition may be between approximately 90 to 110% in embodiments of the present disclosure. It should be appreciated that a pigment having a caramel shade or color may provide an opacity of approximately 100.25%. It should also be appreciated that a pigment having a deep red shade may provide an opacity of approximately 97.09%.

Break Point Measurement

The break point of a lipstick formed according to the formula described hereinabove was measured by using a Cavalla Model #1009 (manufactured by Cavalla Inc. at Hackensack, N.J.). The lipstick, as freshly made, was incubated at 25° C. for 24 hours before testing. The lipstick's cap was removed, and the lipstick was swiveled all the way out and then placed in a holder together with the lipstick case. The gauge needles of the Cavalla were set on zero, and the motor of the Cavalla was turned on. After the lipstick broke transversally, the breakage point was read directly from the black needle points on Cavalla scale. The Cavalla range is between approximately 8-18 units or ounces.

An exploratory clinical was performed to explore the impact, if any, of wax levels on perception of drag and/or texture. A modified pigment formula (MSL4617), which is 40% pigment, free-standing, satin finish lipstick, was used as a reference formula. MSL4617 was compared against a free-standing, satin finish lipstick (MSL3880-1) to obtain feedback on the impact of pigments on the formula. Wax levels were not changed, but different pigments were evaluated with respect to application, texture, and wear.

A comparison of the formulas is shown below in Table 3:

TABLE 3

| Drag | Improved | Initial |
|---|---|---|
| Package | Free | Free |
| | Standing | Standing |
| Pigment | 40% | 40% |

| | | | MSL4617 | MSL3880/1 | |
|---|---|---|---|---|---|
| Type | Purpose | Ingredient | Level | Level | Difference |
| Emollient | Main liquid | Eutanol G | 29.75 | 29.75 | 0.00 |
| | carrier | | | | |
| Pigment | ITT treatment | Red 7CA E-12 | 17.00 | 23.00 | −6.00 |
| Pigment | Non-treated, Iron | Pure Oxy Red | 10.60 | 0.00 | 10.60 |
| | Oxide | 7054/3080 | | | |
| Wax | Structure | Performalene PL | 6.00 | 0.00 | 0.00 |
| Pigment | ITT treatment | Yellow 5AL-12 | 5.50 | 6.90 | −1.40 |
| Pigment | ITT treatment | Red 28AL U-13 | 5.20 | 8.40 | −3.20 |
| Emollient | Feel comfort | Neossance Squalane | 5.00 | 5.00 | 0.00 |
| Wax | Creamy feel not | Multiwax W-835 | 4.00 | 4.00 | 0.00 |
| | structural | | | | |
| Film Former | Long wear | Polyolprepolymer-14 | 3.00 | 3.00 | 0.00 |
| Film Former | Naturally derived, | Unifilma HVY | 3.00 | 3.00 | 0.00 |
| | long wear | | | | |
| Gellant | Structure/stability | Versagel S1000 | 2.00 | 2.00 | 0.00 |
| Wetting | Reduction of | Plurol Diisostearique | 2.00 | 2.00 | 0.00 |
| Agent | surface tension | CG | | | |
| | for better | | | | |
| | pigment/powder | | | | |
| | saturation | | | | |
| Clay | Creamy feel and | Bentone 38 VCG | 1.00 | 1.00 | 0.00 |
| | helps reduce | | | | |
| | bleeding, anti- | | | | |
| | feathering | | | | |
| Silica | Helps reduce | Spheron 20MB | 1.00 | 1.00 | 0.00 |
| | bleeding/anti- | | | | |
| | feathering | | | | |
| Wax | Structure | Ozokerite Wax | 1.00 | 1.00 | 0.00 |
| | | Pastilles 10 | | | |
| Pigment | ITT treatment | BBO-I2 | 0.96 | 0.96 | 0.00 |
| Wetting | Reduction of | Hexaglyn PR-15 | 0.75 | 0.75 | 0.00 |
| Agent | surface tension | | | | |
| | for better | | | | |
| | pigment/powder | | | | |
| | saturation | | | | |
| Pigment | ITT treatment | Red 6SS-12 | 0.74 | 0.74 | 0.00 |
| Moisturizer | Moisturizer | Hyacolor | 0.50 | 0.50 | 0.00 |
| Antioxidant | Conditioner | Vitamin E, USP, | 0.50 | 0.50 | 0.00 |
| | | FCC, Code 0 | | | |
| Moisturizer | Moisturizer | Eldew PS-203 | 0.50 | 0.50 | 0.00 |
| Filler | Translucent for | Barium Sulfate H.L. | 0.00 | 0.00 | 0.00 |
| | truer color | | | | |
| | (shade) | | | | |

Ingredient Type Totals

| | MSL4617 | MSL3880/1 |
|---|---|---|
| Pigment | 40.00 | 40.00 |
| Emollient | 34.75 | 34.75 |
| Wax | 11.00 | 11.00 |
| Film Former | 6.00 | 6.00 |
| Gellant | 5.00 | 2.00 |
| Wetting Agent | 2.75 | 2.75 |
| Clay | 1.00 | 1.00 |
| Silica | 1.00 | 1.00 |
| Moisturizer | 1.00 | 1.00 |
| Antioxidant | 0.50 | 0.50 |
| Filler | 0.00 | 0.00 |

Both formulas were perceived to apply evenly to lips and stay in place upon application; however, MSL4617 outperformed MSL3880-1, garnering favorable scores for being easy to apply and gliding across lips smoothly. The consistency and application of MSL4617 was rated to be just right versus the consistency of MSL3880-1. MSL4617 was found to be easier to apply and control during application, and MSL4617 was found to have less drag than MSL3880-1. Further, while both formulas are perceived as smooth, adjusting the pigments has a positive influence on perception of consistency. In addition, the difference in pigments is positively affecting perception of performance longevity within the wear/appearance benefit space. More specifically, MSL4617 maintained higher, more favorable all-day ratings than MSL3880-1. MSL4617 outperformed MSL3880-1 in several wear-related attributes including maintaining appearance throughout wear, color staying true throughout wear, and being longwearing. The adjustment of pigments within MSL4617 appears to have led to perceptions of easier application with less drag, a more lightweight feeling, a more optimal consistency, positive impact on longevity of performance, most notably sustaining higher scores within the wear/appearance benefit space, higher satisfaction with maintaining its appearance from initial application, and slightly higher satisfaction with the product overall after 8 hours of wear. Thus, the formula modification in MSL4617 contributed positively to perception of ease of application, control during application, product consistency, wear, and sustained overall performance.

An exploratory clinical was performed to conduct pigment load full comparison (40% versus 35% versus 30%). Again, a modified pigment formula (MSL4617) having 40% pigment load was used as a reference. An MSL3892 formula, having a 35% pigment load, and an MSL3880-5 formula, having a 30% pigment load were used. A summary of the formulas is shown below in Table 4:

TABLE 4

| Drag | Improved | Improved | Improved |
|---|---|---|---|
| Package | Free | Free | Free |
| | Standing | Standing | Standing |
| Pigment | 40% | 35% | 30% |

| Type | Purpose | Ingredient | MSL4617 | MSL3892 | MSL3880/5 |
|---|---|---|---|---|---|
| Emollient | Main liquid | Eutanol G | 29.75 | 32.25 | 38.24 |
| | carrier | | | | |
| Pigment | ITT treatment | Red 7CA E-12 | 17.00 | 20.14 | 17.25 |
| Pigment | Non-treated, iron | Pure Oxy Red | 10.60 | 0.00 | 0.00 |
| | oxide | 7054/3080 | | | |
| Wax | Structure | Performalene PL | 6.00 | 5.50 | 5.50 |
| Pigment | ITT treatment | Yellow SAL-12 | 5.50 | 6.03 | 5.18 |
| Pigment | ITT treatment | Red 28AL U-13 | 5.20 | 7.35 | 6.30 |
| Emollient | Feel comfort | Neossance | 5.00 | 5.00 | 5.00 |
| | | Squalane | | | |
| Wax | Creamy feel not | Multiwax W-835 | 4.00 | 4.00 | 4.00 |
| | structural | | | | |
| Film Former | Long wear | Polyolprepolymer- | 3.00 | 3.00 | 3.00 |
| | | 14 | | | |
| Film Former | Naturally | Unifilma HVY | 3.00 | 3.00 | 3.00 |
| | derived, long | | | | |
| | wear | | | | |
| Gellant | Structure/stability | Versagel S1000 | 2.00 | 2.00 | 2.00 |
| Wetting | Reduction of | Plurol | 2.00 | 2.00 | 2.00 |
| Agent | surface tension | Diisostearique CG | | | |
| | for better | | | | |
| | pigment/powder | | | | |
| | saturation | | | | |
| Clay | Creamy feel and | Bentone 38 VCG | 1.00 | 1.00 | 1.00 |
| | helps reduce | | | | |
| | bleeding, anti- | | | | |
| | feathering | | | | |
| Silica | Helps reduce | Spheron 20MB | 1.00 | 1.00 | 1.00 |
| | bleeding/anti- | | | | |
| | feathering | | | | |
| Wax | Structure | Ozokerite Wax | 1.00 | 1.00 | 1.00 |
| | | Pastilles 10 | | | |
| Pigment | ITT treatment | BBO-I2 | 0.96 | 0.84 | 0.72 |
| Wetting | Reduction of | Hexaglyn PR-15 | 0.75 | 0.75 | 0.75 |
| Agent | surface tension | | | | |
| | for better | | | | |
| | pigment/powder | | | | |
| | saturation | | | | |
| Pigment | ITT treatment | Red 6SS-12 | 0.74 | 0.64 | 0.56 |
| Moisturizer | Moisturizer | Hyacolor | 0.50 | 0.50 | 0.50 |
| Antioxidant | Conditioner | Vitamin E, USP, | 0.50 | 0.50 | 0.50 |
| | | FCC, Code 0 | | | |
| Moisturizer | Moisturizer | Eldew PS-203 | 0.50 | 0.50 | 0.50 |
| Filler | Translucent for | Barium Sulfate | 0.00 | 3.00 | 2.00 |
| | truer color | H.L. | | | |
| | (shade) | | | | |

Ingredient Type Totals

| | | | |
|---|---|---|---|
| Pigment | 40.00 | 35.00 | 30.01 |
| Emollient | 34.75 | 37.25 | 43.24 |
| Wax | 11.00 | 10.50 | 10.50 |
| Film Former | 6.00 | 6.00 | 6.00 |
| Gellant | 5.00 | 5.00 | 5.00 |
| Wetting Agent | 2.75 | 2.75 | 2.75 |
| Clay | 1.00 | 1.00 | 1.00 |
| Silica | 1.00 | 1.00 | 1.00 |
| Moisturizer | 1.00 | 1.00 | 1.00 |
| Antioxidant | 0.50 | 0.50 | 0.50 |
| Filler | 0.00 | 3.00 | 2.00 |

Each of these formulas was found to have higher than average scores for ultra-high payoff. Neither a 5% nor a 10% reduction in pigment load was particularly noticeable in terms of color payoff perception. A 10% less pigment load and formula differentiation for MSL3880-5 appears to favorably increase perception of comfort and moisturization. The differences between the formulas and/or pigment loads impact perception of wear differently. The 10% less pigment load and formula differentiation of MSL3880-5 appears to increase perception of bleeding/feathering of the product, while the 5% less pigment load and formula differentiation of MSL3892 appears to negatively impact color being even and uniform, and perception of long-wear generally. Pigment load and formula differentiation does not seem to greatly impact overall perception of transfer resistance though satisfaction with level of transfer was slightly higher with 10% decreased pigment load and formula differentiation of MSL3880-5.

Despite the differences in pigment load and formulation, all formulas generally perform similarly within the Ultra-High Payoff benefit space. All formulas perform similarly through 4 hours for most wear/appearance-related attributes. MSL4617 and MSL3892 perform similarly among comfort-related attributes. MSL3880-5 scores more favorably within this benefit space likely due to formulation difference/10% decreased pigment load. All formulas performed similarly for ratings related to application although MSL3880-5 did achieve slightly more favorable ratings for gliding across lips smoothly. Differences were perceived among the formulas. It appears a 10% difference in pigment load and formulation is advantageous in terms of providing a more ideal perception of comfort even though it does slightly increase perception of bleeding/feathering, though this may potentially be mitigated with use of secondary product (e.g. lip liner). Decreasing pigment load by 5% with formula modifications appears to have not made an impact within comfort space and brought forth performance issues related to wear not previously seen in either 30% or 40% formulas. In general, performance of MSL3892 appears to wane at 6 hours across all benefit spaces (Transfer, High Color Payoff, Wear/Appearance). All formulas performed similarly overall within Ultra-High Payoff space and the pigment load and formula differentiation also did not strongly influence overall performance within Transfer-resistance space.

Another exploratory clinical was performed to evaluate pigment load iteration between the MSL4617 reference formula (40% pigment load) and the MSL3880-5 formula (30% pigment load). Overall, the panel perceived a difference in the formulas as the 30% pigment load was found to be less dry. The 10% difference in pigment load did not impact coverage, and it also did not affect perception of color impact. Both formulas performed similarly within the ultra-high payoff space and across other wear-related attributes. The pigment load and formula differentiation did not strongly influence performance within the transfer-resistance space although satisfaction with level of transfer was slightly higher with decreased pigment load. The 10% less pigment load in MSL3880-5 appeared to favorably increase perception of comfort and moisturization, but it also increased perception of bleeding/feathering of the product.

All percentages, parts and ratios are based upon the total weight of the compositions of the present disclosure, unless otherwise specified. All such weights as they pertain to listed ingredients are based on the active level, and therefore they do not include solvents or by-products that may be included in commercially available materials, unless otherwise specified. All molecular weights, as used herein, are weight average molecular weights expressed as grams/mole, unless otherwise specified.

The term “comprising” and the like, means that a list of elements may not be limited to those explicitly recited. The term “weight percent” may be denoted as “wt. %” herein. The term “long wear” or the like refers to compositions of the present disclosure that are not readily removed by contact with another material, such as clothing or water. Long wear may be evaluated by any method known in the art. For example, a composition may be evaluated based on the amount of product remaining on the skin or hair of a wearer after minutes or hours of wear. For example, a composition may provide long wear attributes if a majority of the product is left on the wearer's skin or hair after minutes or hours of wear. Further, the amount remaining on the wearer's skin or hair may be compared with that transferred by other compositions, such as commercially available compositions. In preferred embodiments of the present disclosure, little or no composition is removed from the skin or hair after minutes or hours of wear.

The term “pigment” or the like refers to ingredients including, but not limited to, colorant ingredients, a complex, powders, and/or fillers that may be surface treated with, for example, isopropyl titanium triisostearate (ITT), trityl tetrazole bromomethyl biphenyl (TTBB2), silane, and/or dimethicone. Pigment also may refer to ingredients including, but not limited to, bentonite, hectorite, silica, hexamethylene diisocyanate (HDI)/trimethylol hexyllactone crosspolymer and silica (BPD-500), mica, barium, and/or sulfate.

The term “opacity” or the like refers to the transparency or contrast ratio of the cosmetic composition. A higher opacity may correspond to a higher pigment load, thereby providing greater coverage to a wearer of the cosmetic composition. The term “ultra-saturated color deposition” or the like refers to the greatest level of liquid vehicle uptake in the pigment structure due to complete wetting through chemical and mechanical means.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “approximately 40 mm.”

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of making a cosmetic composition, the method comprising:
mixing, in a pre-dispersion phase, at least 35% by weight of the cosmetic composition of one or more solid particulates with liquid ingredients,
wherein the one or more solid particulates comprise pigments, powders, and fillers, and
wherein the liquid ingredients include approximately 29-42% by weight of the cosmetic composition of one or more emollients and 0.25-5% by weight of the cosmetic composition of one or more wetting agents.

2. The method of claim 1, the liquid ingredients further comprising:
0.5-10% by weight of one or more film formers;
0.5-1% by weight of vitamin E;
2-3% by weight of a copolymer block;
1-2% by weight of clay; and
1-2% by weight of silica.

3. The method of claim 1, wherein the one or more solid particulates fully saturate the liquid ingredients and fully disperse in forming the cosmetic composition.

4. The method of claim 1 further comprising:
grinding, in a grind phase, the cosmetic composition until particles have less than approximately 12 microns or approximately 7 Hegman units.

5. The method of claim 4 further comprising:
mixing, in a full extender phase, the ground cosmetic composition with one or more of the following:
one or more emollients, one or more wetting agents, one or more waxes, one or more film formers, and one or more moisturizers.

6. The method of claim 1 further comprising:
heating the cosmetic composition up to approximately 95 degrees Celsius.

7. A cosmetic composition comprising:
one or more solid particulates;
at least one of a dispersing agent, a wetting agent, or a mixture thereof,
at least one film former; and
at least one main carrier.

8. The cosmetic composition of claim 7 comprising:
approximately 35-50% by weight of the one or more solid particulates;
approximately 0.25-5% by weight of the at least one of the dispersing agent, the wetting agent, or the mixture thereof;
approximately 0.5-10% by weight of the at least one film former; and
approximately 35% by weight of the at least one main carrier.

9. The cosmetic composition of claim 7 comprising:
a. at least 35% by weight of the one or more solid particulates;
b. at least 0.25% by weight of the at least one of the dispersing agent, the wetting agent, or the mixture thereof;
c. at least 0.50% by weight of the at least one film former;
d. at least 5% by weight of one or more wax-related components; and
e. at least 0.25% by weight of one or more moisturizing agents.

10. The cosmetic composition of claim 7, wherein the one or more solid particulates is selected from the group consisting of pigments, powders, fillers, and mixtures thereof.

11. The composition of claim 7, wherein the one or more solid particulates are surface treated to provide a shear-resistant composition.

12. The composition of claim 7, wherein the main carrier is selected from one or more of long-chain fatty alcohol liquids, oils, esters, and emollients.

13. The composition of claim 7, wherein the cosmetic composition provides ultra-saturated color deposition when applied to a user, wherein the ultra-saturated color deposition provides at least 70% opacity.
